(19) United States
(12) Patent Application Publication
(10) Pub. No.: US 2004/0044075 A1
(43) Pub. Date: Mar. 4, 2004

(54) Alpha acyloxyacetamides for kallikrein and urokinase inhibition

(75) Inventors: Mark M. Staveski, Taunton, MA (US); Scott F. Sneddon, Salem, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genzyme Corporation, Cambridge, MA

(21) Appl. No.: 10/448,447

(22) Filed: May 29, 2003

Related U.S. Application Data
(60) Provisional application No. 60/385,264, filed on May 31, 2002.

(51) Int. Cl. A61K 31/24

(52) U.S. Cl. 514/540

(57) ABSTRACT

Disclosed herein is a compound represented by Structural Formula (I):

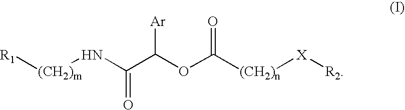

R₁ is a substituted or unsubstituted aryl group or alkyl group; R₂ is a substituted or unsubstituted aryl group or cycloalkyl group; Ar is a substituted or unsubstituted aryl group; X is a —CH₂—, —O—, —S— or —CO—; m is an integer from zero to two; n is an integer from 0-2 when X is —O—, —S— and 1-2 when X is —CH₂— or —CO—.

Also disclosed are methods of inhibiting kallikrein activity or urokinase activity in subject in need of such inhibition by administering a compound represented by Structural Formula (I).

### RELATED APPLICATION

This application claims the benefit of U.S. Provisional Application No. 60/385,264, filed May 31, 2002. The entire teachings of the above application is incorporated herein by reference.

### BACKGROUND OF THE INVENTION

Human cancers generally require proteolytic enzymes to invade adjacent tissues and form metastases. The formation of new blood vessels is typically required for tumor growth. Urokinase (uPA), a member of the serine protease family, promotes tumor cell migration and invasion by converting the inactive zymogen plasminogen into the active serine protease, plasmin, which then cleaves extracellular matrix components including laminin, fibronectin and collagen. This process also promotes angiogenesis, the formation of new blood vessels, through the release of matrix-bound growth factors such as VEGF and bFGF. It has been shown that inhibition of uPA can decrease tumor size or even cause complete remission of cancers in mice. New and more potent inhibitors of urokinase are therefore needed for the treatment of human cancers.

Plasma kallikrein is responsible for cleaving kininigen to form bradykinin, a mediator of pain and inflammation. Bradykinin has been implicated in inflammatory diseases such as inflammatory bowel disease, rheumatoid arthritis, asthma and allergic rhinitis. Inhibitors of the kallikrein enzyme can provide clinical benefits by preventing production of bradykinin. In addition, kallikrein has been found to activate urokinase.

Thus, inhibitors of kallikrein should be effective against urokinase and therefore can also be used as treatments against cancers. Thus, new inhibitors of kallikrein are needed for the treatment of pain, inflammatory diseases and cancer.

### SUMMARY OF THE INVENTION

It has now been found that certain alpha aryl acyloxyacetamides are potent inhibitors of kallikrein and urokinase. For example, many of the alpha aryl acyloxyacetamides shown in Tables 1 and 5 have an IC₅₀ less than 5.0 μM, and some less than 1.0 μM for the inhibition of kallikrein or urokinase (see Examples 5 and 9). Moreover, the alpha phenyl acyloxyacetamides shown in Tables 2 and 3 demonstrated analgesic activity when tested in mice with an acetic acid writhing assay (see Example 6) and/or anti-inflammatory activity when tested in rats with a carageenan assay (see Example 7); and Compound 26 had activity in a rat model of inflammatory bowel disease (Example 8). Based on these discoveries, novel alpha aryl acyloxyacetamides, methods of treating a subject with an inflammatory disorder and methods of treating a subject with cancer are disclosed herein.

One embodiment of the present invention is a compound represented by Structural Formula (I):

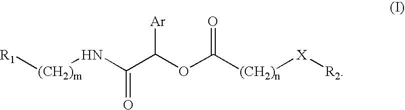

R₁ is a substituted or unsubstituted aryl group or alkyl group.

R₂ is a substituted or unsubstituted aryl group or cycloalkyl group.

Ar is a substituted or unsubstituted aryl group.

X is a —CH₂—, —O—, —S— or —CO.

m is an integer from zero to two.

n is an integer from 0-2 when X is —O—, —S— or 1-2 when X is —CH₂— or —CO—.

In one aspect, when Ar is a substituted or unsubstituted phenyl group and X is —CH₂— in Structural Formula (T), then R₁ is a cyclopropyl group or R₂ is a substituted or unsubstituted indolyl, pyrimidinyl, benzothienyl, cyclopentyl or cyclohexyl group.

Another embodiment of the present invention is a method of inhibiting kallikrein activity in a subject in need of a such inhibition. The method comprises administering to the subject an effective amount of a compound represented by Structural Formula (I).

Yet another embodiment of the present invention is a method of inhibiting urokinase activity in a subject in need of a such inhibition. The method comprises administering to the subject an effective amount of a compound represented by Structural Formula (I).

The compounds disclosed herein are effective inhibitors of kallikrein and/or urokinase and thus are useful as analgesics, in the treatment of cancer and/or the treatment of inflammatory diseases.

### DETAILED DESCRIPTION OF THE INVENTION

The present invention is directed to compounds represented by Structural Formula (I) and the use of such compounds for inhibiting kallikrein activity and/or urokinase activity in a subject in need of such treatment. Definitions of the terms used to describe these inventions are provided below.

The term “aryl group”, e.g., the aryl groups represented by R₁, R₂ and Ar, refers to carbocyclic aromatic groups such as phenyl, naphthyl, and anthracyl, and heteroaryl groups such as imidazolyl, isoimidazolyl, thienyl, furanyl, pyridyl, pyrimidyl, pyranyl, pyrazolyl, pyrrolyl, pyrazinyl, thiazolyl, isothiazolyl, oxazolyl, isooxazolyl, 1,2,3-triazolyl, 1,2,4-triazolyl, and tetrazolyl.

Aryl groups, such as the aryl groups represented by R₁, R₂ and Ar, also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

An alkyl group is a straight, branched or cyclic non-aromatic hydrocarbon which is completely saturated or which contains one or more units of unsaturation. Typically, a straight or branched alkyl group has from 1 to about 10 carbon atoms, preferably from 1 to about 4, and a cyclic aliphatic group has from 3 to about 10 carbon atoms, preferably from 3 to about 8. Examples of suitable straight or branched alkyl group include methyl, ethyl, n-propyl, iso-propyl, n-butyl, sec-butyl, tert-butyl, pentyl, hexyl, heptyl or octyl; and examples of suitable cycloalkyl groups include cyclopropyl, cyclobutyl, cyclopentyl, cyclohexyl, cycloheptyl and cyclooctyl. A C1-C10 straight or branched alkyl group or a C3-C8 cyclic alkyl group are also referred to as a “lower alkyl” group.

Suitable substituents for an alkyl group, an aryl group (e.g., the aryl group represented by R₁, R₂ and Ar) or a non-aromatic heterocyclic group are those which do not substantially interfere with the ability of the compound to inhibit kallikrein and/or urokinase activity. Examples of suitable substituents for any aryl (e.g., the aryl group represented by R₁, R₂ or Ar), alkyl or non-aromatic heterocyclic group include —OH, halogen (—Br, —Cl, —I and —F), —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, NHCONR^{a}H, —NHCON(R^{a}R^{b}), —NR^{c}CONH₂, —NR^{c}CONR^{a}H, —NR^{c}CON(R^{a}R^{b}), —C(—NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), _NR^{d}H—C(═NH)—NH₂, _NR^{d}—C(═NH)—NHR^{a}, _NR^{d}—C(═NH)N(R^{a}R^{b}), N(R^{a}R^{b}) —NR^{d}—C(═NR^{c})—NH₂, —NR^{d}—C(═NR^{c})-NHR^{a}, —NR^{d}—C(═NR^{c})-N(R^{a}R^{b}), —SO₂NH₂, —SO₂NHR^{a}, —SO₂NR^{a}R^{b}, —SH, —SOₖR^{a} (k is 0, 1 or 2) and —NH—C(═NH)—NH₂. R^{a}-R^{d} are each independently an alkyl, substituted alkyl, benzyl, substituted benzyl, aryl or substituted aryl group, preferably an alkyl, benzylic or aryl group. In addition, —NR^{a}R^{d}, taken together, can also form a substituted or unsubstituted non-aromatic heterocyclic group, such as pyrollidinyl, piperidinyl, morpholinyl and thiomorpholinyl. A substituted aryl (including the aryl groups represented by R₁, R₂ and Ar), alkyl and non-aromatic heterocyclic group can have more than one substitutent.

Preferred substituents for an aryl group (e.g., the aryl group represented by R₁, R₂ and Ar) include methylenedioxy, —CO—NH₂, —O(C1-C4 alkyl), —F, —Cl, —Br, —CN, C1-C4 alkyl, C1-C4 haloalkyl, —CF₃, —SO₂(C1-C4 alkyl), —COO(C1-C4 alkyl) and —S(C1-C4 alkyl). For example, zero, one or more of these substituents are typically present when R₁ is an optionally substituted phenyl group, Ar is an optionally substituted phenyl or naphthyl group and R₂ is an optionally substituted phenyl or pyrimidyl group.

Particularly preferred substituents for a phenyl group represented by R₁ and R₂ are methoxy or methylenedioxy, for example, 3,4-dimethoxyphenyl or 3,4,5-trimethoxyphenyl, 3,4-methylenedioxy for R₁ and monosubstituted phenyl (e.g., 4-methoxyphenyl) and disubstituted phenyl (e.g., 2,5-dimethoxyphenyl) for R₂. Ar is typically substituted at the 3, 4 and/or 5 position; cyano and bromo are particularly preferred as substitutents for a phenyl group represented by Ar.

Preferred substituents for an alkyl group include —O(C1-C4 alkyl), —S(C1-C4 alkyl), —COO(C1-C4 alkyl).

Also included in the present invention are pharmaceutically acceptable salts of the compounds described herein. Compounds disclosed herein which possess a sufficiently acidic, a sufficiently basic, or both functional groups, and accordingly can react with any of a number of organic or inorganic bases, and inorganic and organic acids, to form a salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The disclosed compounds can be used to inhibit kallikrein activity in a subject in need of such treatment. Subjects “in need of kallikrein inhibition” are those with a disease, condition or abnormality resulting from excessive kallikrein activity or insufficient kallikrein inhibition. Subjects “in need of kallikrein inhibition” also include those for whom a beneficial therapeutic or prophylactic effect can be achieved by kallikrein inhibition. For example, kallikrein cleaves kinnigen to form bradykinnin, a mediator of pain and inflammation. Therefore, the disclosed compounds can be used as analgesics and to treat, inter alia, subjects with inflammatory diseases such as inflammatory bowel disease (e.g., colititis and Crohn's disease) and rheumatoid arthritis. Because kallikrein activates urokinase activity, the kallikrein inhibitors disclosed herein can also be used to inhibit urokinase activity and thus be used in the treatment of cancer.

The disclosed compounds can also be used to inhibit urokinase activity in a subject in need such treatment. Subjects “in need of urokinase inhibition” are those with a disease, condition or abnormality resulting from excessive urokinase activity or insufficient urokinase inhibition. Subjects “in need of urokinase inhibition” also include those for whom a beneficial therapeutic or prophylactic effect can be achieved by urokinase inhibition. For example, urokinase inhibition has been shown to be useful in the treatment of cancer and in the inhibition of angiogenesis. Because urokinase inhibition modulates growth factor release from extra-cellular matrix, the disclosed urokinase inhibitors are also expected to act as immunomodulators.

An “effective amount” is the quantity of compound in which a beneficial clinical outcome (prophylactic or therapeutic) is achieved when the compound is administered to a subject in need of treatment. For the treatment of inflammatory disorders, a “beneficial clinical outcome” includes a reduction in the severity of the symptoms associated with the disease (e.g., pain and inflammation), an increase in the longevity of the subject and/or a delay in the onset of the symptoms associated with the disease compared with the absence of the treatment. For the treatment of cancer, a beneficial clinical outcome includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. Effective amounts of the disclosed compounds typically range between about 0.1 mg/kg body weight per day and about 1000 mg/kg body weight per day, and preferably between 1 mg/kg body weight per day and 100 mg/kg body weight per day.

The disclosed compounds are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), or rectally, depending on the type of cancer to be treated. Oral or parenteral administration are preferred modes of administration.

The disclosed compounds can be administered to the subject in conjunction with an acceptable pharmaceutical carrier as part of a pharmaceutical composition for treatment of cancer. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al, “Controlled Release of Biological Active Agents”, John Wiley and Sons, 1986).

When used to treat inflammatory diseases, the disclosed compounds can be coadministered with other analgesics and/or anti-inflammatory agents, such as aspirin, acetaminophen, REMICADE or a COX2 inhibitor. When used to treat cancer, the disclosed compounds can be advantageously used in combination with other anti-cancer therapy, including drugs (e.g., taxol, flurouracil, methatrexate, cisplatin, and the like), radiation and/or surgery.

A “subject” is a mammal, preferably a human, but can also be an animal in need of veterinary treatment, e.g., companion animals (e.g., dogs, cats, rabbits and the like), farm animals (e.g., cows, sheep, pigs, horses, and the like) and laboratory animals (e.g., rats, mice, guinea pigs, and the like).

Compounds represented by Structural Formula (I), wherein m is 0, can be prepared by reacting an aryl aldehyde (ArCHO), an aryl isocyanide (R₁N⁺═C⁻) and an aryl alkanoic acid, as shown below in Scheme I:

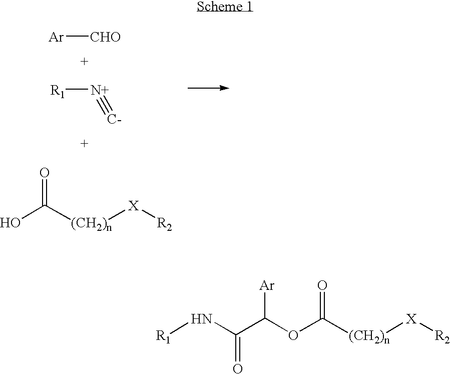

R₁, R₂, Ar, X and n in Scheme 1 defined as in Structural Formula (I). The reaction is carried out by mixing the reagents together in a suitable solvent, for example, an alcoholic solvent. Optionally, the reaction can be heated to bring about more rapid product formation, for example, from about 40° C. to about 70° C. Illustrative conditions for carrying out the reaction are provided in Examples 1 and 3.

Another method for preparing the compounds of the present invention is to amidate an O-protected aryl mandelic acid with a suitable amine and then remove the O-protecting group. Amidation can be carried out by a variety of methods known to one of ordinary skill in the art, including first converting the carboxylic acid group to an acid chloride with thionyl chloride or oxalyl chloride, for example, and then reacting the acid chloride thus formed with the amine. The free alcohol group is then used to esterify a suitable alkanoic acid using a carboxylic acid activating agent such as dicyclohexylcarbodiimide, 1-(3-dimethylaminopropyl)-3-ethylcarbodimide hydrochloride (EDCI) or other coupling reagents commonly known or typically used in peptide synthesis. Esterification reactions of this type are also known to one of ordinary skill in the art. This reaction sequence is illustrated in Scheme (2) below:

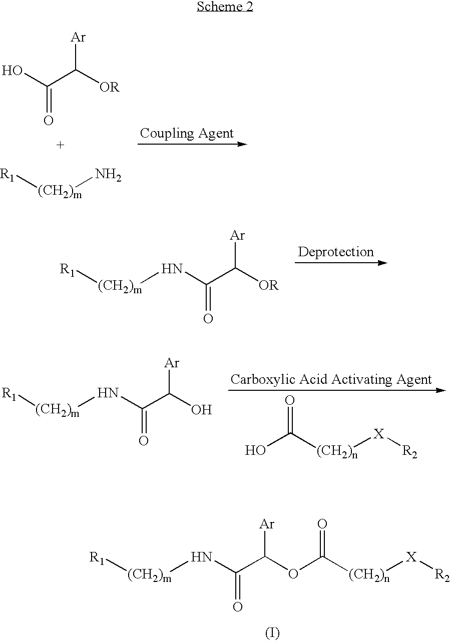

R₁, R₂, Ar, X, n and m in Scheme 2 are as defined in Structural Formula (I); and R is a hydroxyl protecting group such as acetyl. Illustrative conditions for carrying out these reactions are provided in Examples 2 and 4.

In one preferred embodiment, the compound of the present invention is represented by Structural Formula (II):

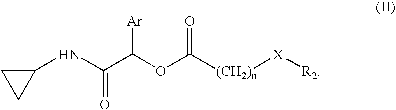

The variables in Structural Formula (II) are as defined above for Structural Formula (I). Preferably, Ar is a substituted or unsubstituted phenyl or naphthyl group.

When the compound represented by Structural Formula (II) is being used to inhibit kallikrein activity in a subject, then R₂ in Structural Formula (II) is preferably a substituted or unsubstituted phenyl, cyclohexyl or indolyl group. More preferably, n is 0, X is —CH₂—, and R₂ is an optionally substituted phenyl or indolyl group. Alternatively, n is 2, X is —CO— and R₂ is a substituted or unsubstituted phenyl group. Specific examples of kallikrein inhibitors represented by Structural Formula (II) are shown below as Compounds (1)-(3):

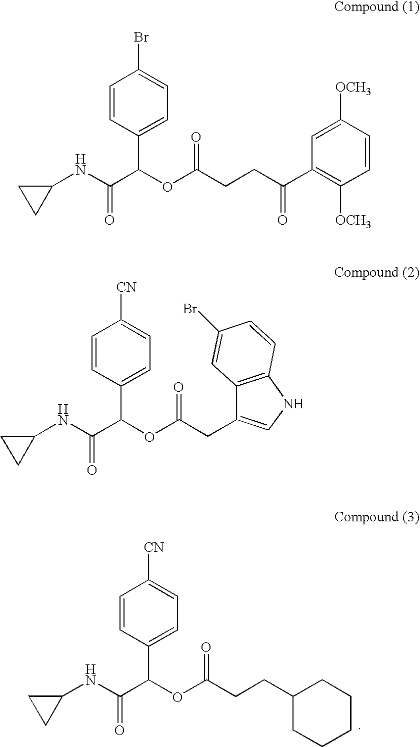

Other specific examples of kallikrein inhibitors represented by Structural Formula (II) are shown in Table 1 of Example 5.

When the compound represented by Structural Formula (II) is being used to inhibit urokinase activity in a subject, R₂ in Structural Formula (II) is preferably a substituted or unsubstituted phenyl, C5-C6 cycloalkyl, pyrimidyl or indolyl group. More preferably, n is 0, X is —CH₂—, and R₂ is an optionally substituted phenyl or indolyl group (e.g., 4-methoxyphenyl, 3-indolyl or 5-bromo-3-indolyl). Alternatively, n is 1, X is —S—, and R₂ is an optionally substituted pyrimidyl group (e.g., 2-pyrimidyl). In another alternative, n is 1, X is —CH₂— and R₂ is a cyclopentyl or cyclohexyl group. Specific examples of urokinase inhibitors represented by Structural Formula (II) are shown below as Compounds (4)-(7):

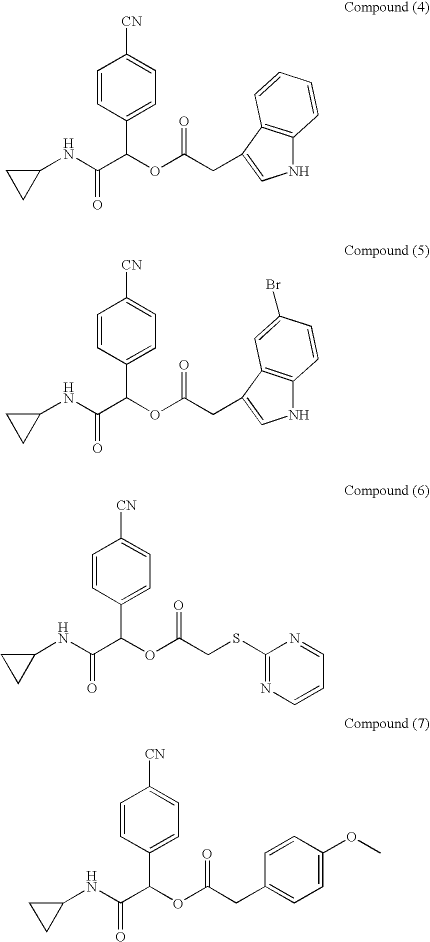

Other specific examples of urokinase inhibitors represented by Structural Formula (II) are shown in Table 5 of Example 9.

In another preferred embodiment, the compound of the present invention is represented by Structural Formula (III) or (IV):

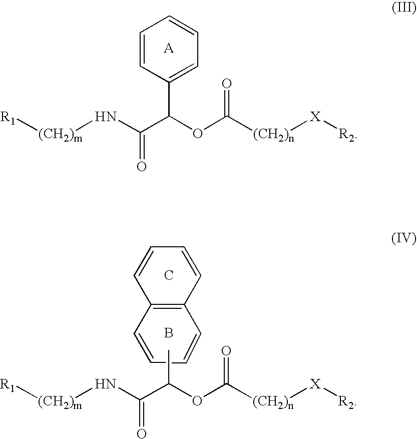

The variables in Structural Formulas (III) and (TV) are as described in Structural Formula (I) above. Phenyl Rings A-C are substituted or unsubstituted.

When the compound represented by Structural Formula (III) or (IV) is being used to inhibit kallikrein activity, R₁ is preferably a substituted or unstubstituted phenyl group. More preferably, R₁ is a substituted or unstubstituted phenyl group and R₂ is a substituted or unsubstitutted phenyl, indolyl, pyrimidyl or benzothienyl group. Even more preferably, R₁ and R₂ are as defined above in Structural Formula (I) (e.g., a phenyl group optionally substituted with one or more methoxy groups), m is 0 or 1; n is 2 and X is —CO—. Kallikrein inhibitors that are particularly preferred are represented by Structural Formula (V):

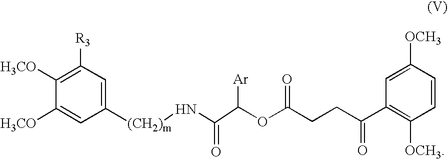

In Structural Formula (V), m is 0 or 1; R₃ is —H or —OCH₃; and Ar is naphthyl or phenyl optionally monosubstituted with —CN or —Br (e.g., 2-napthyl, 4-bromophenyl or 3-cyanophenyl).

Specific examples of kallikrein inhibitors represented by Structural Formulas (III)-(V) are shown in Table 1 of Example 5.

When the compound represented by Structural Formulas (III) and (IV) is being used to inhibit urokinase, R₁ in one preferred embodiment is a substituted or unsubstituted alkyl group (e.g., C1-C10 alkyl group, preferably C1-C4 alkyl group). More preferably m is 0; n is 0 or 1; X is —CH₂— or —S—; the alkyl represented by R₁ has from one to four carbon atoms and is optionally substituted with —O(C1-C4 alkyl), —S(C1-C4 alkyl) or —COO(C1-C4 alkyl); Ar is an optionally substituted phenyl group; and R₂ is an optionally substituted phenyl or pyrimidyl group.

When the compounds represented by Structural Formulas (III) and (IV) are used to inhibit urokinase, R₁ in another preferred embodiment is a substituted or unsubstituted phenyl group. When R₁ is a substituted or unstubstituted phenyl group, R₂ is preferably a substituted or unsubstituted pyrimidyl or phenyl group. More preferably, R₁ and R₂ are as described above and n is 0 or 1; X is —CH₂— or —S—; and m is 1 or 2. For preferred urokinase inhibitors represented by Structural Formulas (III) or (IV), R₁ is a phenyl group substituted with one or more methoxy groups (e.g., 4-methoxyphenyl, 3,4-dimethoxyphenyl or 3,4,5-trimethoxyphenyl group) and/or methylenedioxy groups (e.g., 3,4-methlyenedioxyphenyl); Ar is a phenyl group optionally substituted with a halogen or cyano group (e.g., phenyl, 3-bromo, 4-bromo, 3-cyano or 4-cyanophenyl); and R₂ is a pyrimidyl group (e.g., 2-pyrimidyl group) or phenyl group monosubstituted with a methoxy group. Variables n, m and X are as defined in the prior paragraph. Specific examples of urokinase inhibitors represented by Structural Formula (III) are shown below as Compounds (8)-(10):

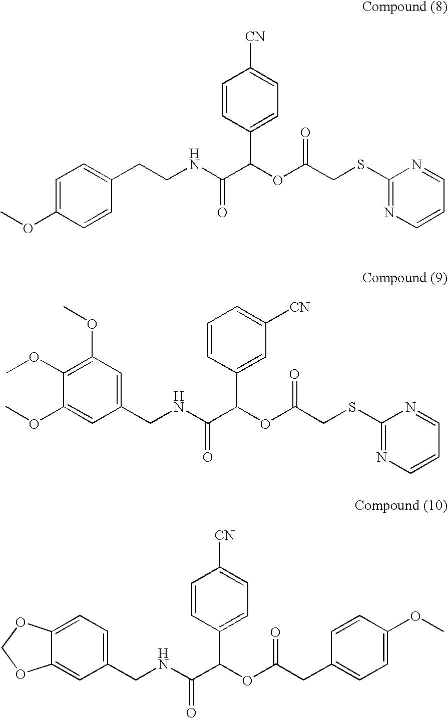

Other specific examples of urokinase inhibitors represented by Structural Formulas (IV) and (V) are shown in Table 5 of Example 9.

Another embodiment of the present invention is a compound represented by Structural Formula (I), (II), (III) or (IV), wherein R₁ corresponds to the group at the corresponding position in any one of the compounds shown in Tables 1 and 5. Also included is a method utilizing such compounds for kallikrein and/or urokinase inhibition in a subject, as described herein.

Another embodiment of the present invention is a compound represented by Structural Formula (I), (II), (III) or (IV), wherein R₂ corresponds to the group at the corresponding position in any one of the compounds shown in Tables 1 and 5. Also included a method utilizing such compounds for kallikrein and/or urokinase inhibition in a subject, as described herein.

Another embodiment of the present invention is a compound represented by Structural Formula (I), (II), (III) or (IV), wherein R₁ and R₂ are each selected to correspond to the group at the corresponding position of any one of the compounds shown in Tables 1 and 5. Also included is a method utilizing such compounds for kallikrein and/or urokinase inhibition in a subject, as described herein.

Another embodiment of the present invention is a compound represented by Structural Formula (I), (II), (III) or (IV), wherein R₁ and Ar are each selected to correspond to the group at the corresponding position of any one of the compounds shown in Tables 1 and 5. Also included is a method utilizing such compounds for kallikrein and/or urokinase inhibition in a subject, as described herein.

Another embodiment of the present invention is a compound represented by Structural Formula (I)-(IV), wherein Ar and R₂ are each selected to correspond to the group at the corresponding position of any one of the compounds shown in Tables 1 and 5. Also included is a method utilizing such compounds for kallikrein and/or urokinase inhibition in a subject, as described herein.

The invention is illustrated by the following examples which are not intended to be limiting in any way.

### EXEMPLIFICATION

### Example 1

### Preparation of (4-Methoxy-Phenyl)-Acetic Acid (4-Cyano-Phenyl)-(3,4-Dimethoxy-Phenylcarbamoyl)-Methyl Ester

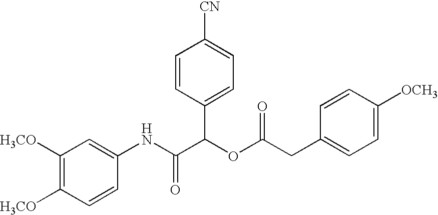

4-Cyanobenzaldehyde (52 mg, 0.40 mmole), 4-methoxyphenylacetic acid (70 mg, 0.44 mmole) and 3,4-dimethoxyphenyl isocyanide (64 mg, 0.40 mmole) were dissolved in 5 ml methanol and warmed to 50° C. The mixture was allowed to stir at this temperature overnight. The reaction was worked up by concentrating in-vacuo and dissolving the residue in 10 ml ethyl acetate. This was then washed with 1N HCl, saturated sodium bicarbonate, and saturated sodium chloride, dried over Na₂SO₄ and concentrated in-vacuo. The compound was then purified by flash chromatography on silica gel (1:1 ethyl acetate/hexane). ¹H NMR (300 MHz, CDCl₃) δ 7.64 (d, 2H), 7.54 (d, 2H), 7.42 (bs, 1H), 7.30 (d, 2H), 7.11 (d, 1H), 6.95 (d, 2H), 6.75 (d, 1H), 6.58 (dd, 1H), 6.21 (s, 1H), 3.85 (s, 3H), 3.83 (s, 3H), 3.81 (s, 3H), 3.77 (s, 2H).

### Example 2

### Preparation of 4-(2,5-Dimethoxyphenyl)-4-Oxo-Butyric Acid (4-Bromophenyl)-(3,4,5-Trimethoxy Benzylcarbamoyl)-Methyl Ester

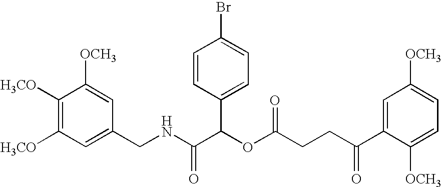

Step 1—Preparation of Acetoxy-(4-bromophenyl)-acetic Acid

Bromomandelic acid (10.30 g, 0.045 mole) was dissolved in 25 ml pyridine. Acetic anhydride (5.01 g, 0.050 mole) was added dropwise to this mixture over 15 minutes. The reaction was allowed to stir at room temperature overnight. The reaction was concentrated in-vacuo and dissolved in 75 ml ethyl acetate. This was then washed with 1 N HCl and saturated sodium chloride, dried over MgSO₄ and concentrated in-vacuo to give a colorless oil. ¹H NMR (300 MHz, CDCl₃) δ 9.28 (bs, 1H), 7.56 (d, 2H), 7.38 (d, 2H), 5.88 (s, 1H), 2.19 (s, 3H).

Step 2 Preparation of Acetic Acid (4-bromophenyl)-(3,4,5-trimethoxybenzylcarbamoyl)-methyl Ester

To the product of step 1 (6.10 g, 0.022 mole), dissolved in 30 ml dichloromethane, was added dimethylformamide (0.1 ml, catalytic). The mixture was cooled in an ice water bath to 0° C. and oxalyl chloride (16.5 ml, 0.033 mole) was added drop-wise over 15 minutes. The reaction mixture was allowed to warm to room temperature and stirred for 1 hour. The reaction was then concentrated in-vacuo and dried under vacuum. The acid chloride was dissolved in 20 ml dichloromethane and then added over fifteen minutes to a solution of 3,4,5-trimethoxybenzylamine (4.84 g, 0.025 mole) and triethylamine (2.48 g, 0.025 mole) at 0° C. The reaction was then allowed to warm to room temperature and stirred for 4 hours. The reaction was diluted with 30 ml dichloromethane and this solution was then washed twice with 1M HCl, twice with saturated sodium bicarbonate and twice with saturated sodium chloride. The organic layer was then dried over MgSO₄ and concentrated in-vacuo to give a golden foamy solid. ¹H NMR (300 MHz, CDCl₃) δ 7.41 (d, 2H), 7.30 (d, 2H), 6.90 (bt, 1H), 6.28 (s, 2H), 5.92 (s, 1H), 4.26 (m, 2H), 3.77 (s, 3H), 3.63 (s, 6H), 2.10 (s, 3H).

Step 3-Preparation of 2-(4-bromophenyl)-2-hydroxy-N-(3,4,5-trimethoxybenzyl)-acetamide

To the amide-acetate from step 2 (7.90 g, 0.0175 mole), dissolved in 40 ml tetrahydrofuran, was added lithium hydroxide monohydrate (1.10 g, 0.0262 mole) dissolved in 15 ml water. The resulting mixture was stirred at room temperature. After 2 hours, 10 ml water and 30 ml diethyl ether were added, resulting in the precipitation of a white solid. The solid was filtered and washed with diethyl ether. The filtrate was then collected and the organic layer was separated and concentrated in-vacuo to one-third volume. Additional solid precipitated, which was collected, as described above. The combined solid was dried in a vacuum oven at 40° C. for 4 hours. ¹H NMR (300 MHz, CDCl₃) δ 7.48 (d, 2H), 7.31 (d, 2H), 6.50 (bt, 1H), 6.32 (s, 2H), 5.06 (s, 1H), 4.38 (m, 2H), 3.81 (s, 3H), 3.76 (s, 6H).

Step 4—Preparation of 4-(2,5-Dimethoxyphenyl)-4-oxo-butyric acid (4-bromophenyl)(3,4,5-trimethoxy-benzylcarbamoyl)-methyl Ester

The hydroxy amide from step 3 (4.10 g, 0.010 mole) and 2,5-dimethoxybenzoyl propionic acid (2.50 g, 0.011 mole) were mostly dissolved 125 ml dichloromethane. To this reaction was added dimethylaminopyridine (0.122 g, 0.001 mole) and then EDCI (3.30 g, 0.011 mole) dissolved in 25 ml dichloromethane. The reaction was allowed to stir at room temperature for 16 hours. The reaction mixture was then transferred to a separatory funnel and washed twice with 1M HCl, twice with saturated sodium bicarbonate and twice with saturated sodium chloride. The organic layer was then dried over MgSO₄ and concentrated in-vacuo. The compound was then purified by flash chromatography on silica gel eluted with 2:1 ethyl acetate/hexane. ¹H NMR (300 MHz, CDCl₃) δ 7.55 (bt, 1H), 7.49 (d, 2H), 7.38 (d, 2H), 7.18 (d, 1H), 7.05 (dd, 1H), 6.92 (d, 1H), 6.42 (s, 2H), 6.09 (s, 1H), 4.46 (m, 2H), 3.87 (s, 3H), 3.76 (s, 3H), 3.73 (s, 6H), 3.69 (s, 3H), 3.45 (m, 2H), 2.84 (m, 1H), 2.66 (m, 1H).

### Example 3

### Preparation of 4-Methoxyphenyl Acetic Acid [(Benzo[1,3] Dioxol-5-ylmethyl)-Carbamoyl](4-Cyanophenyl) Methyl Ester

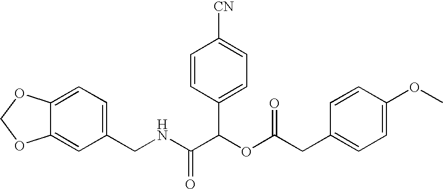

4-Cyanobenzaldehyde (131 mg, 1.00 mmole), 4-methoxyphenylacetic acid (166 mg, 1.00 mmole) and 3,4-methylenedioxybenzyl isocyanide (161 mg, 1.00 mmole) were dissolved in 25 ml methanol and warmed to 600 C. The reaction was allowed to stir at this temperature overnight. The reaction was then worked up by concentrating in-vacuo and dissolving the residue in 50 ml ethyl acetate. This was then washed with 1N HCl, saturated sodium bicarbonate, and saturated sodium chloride, dried over Na₂SO₄ and concentrated in-vacuo. The compound was then purified by flash chromatography on silica gel (1:1 ethyl acetate/hexane). ¹H NMR (300 MHz, CDCl₃) δ 7.61 (d, 2H), 7.48 (d, 2H), 7.15 (d, 2H), 6.80 (d, 2H), 6.74 (d, 1H), 6.58 (d, 2H), 6.14 (bs, 1H), 6.12 (s, 1H), 5.96 (s, 2H), 4.10 (d, 2H), 3.77 (s, 3H), 3.69 (s, 2H).

### Example 4

### Preparation of 3-(Cyclohexyl)-Propionic Acid (4-Cyanophenyl)(Cyclopropylcarbamoyl)-Methyl Ester

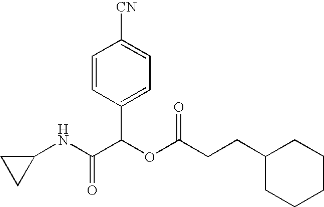

Step 1—Preparation of Acetoxy-(4-cyanophenyl)-acetic Acid

4-Cyanomandelic acid (8.90 g, 0.050 mole) was dissolved in 50 ml pyridine. Acetic anhydride (6.15 g, 0.060 mole) was then added dropwise to this solution over 15 minutes. The reaction was then allowed to stir at room temperature overnight. The reaction was then concentrated in-vacuo and dissolved in 100 ml dichloromethane. This solution was then washed with 1N HCl (2×) and saturated sodium chloride, dried over MgSO₄ and concentrated in-vacuo to give a colorless oil. ¹H NMR (300 MHz, CDCl₃) δ 8.94 (bs, 1H), 7.69 (d, 2H), 7.61 (d, 2H), 5.98 (s, 1H), 2.21 (s, 3H).

Step 2—Preparation of Acetic Acid (4-cyanophenyl)-(cyclopropylcarbamoyl)-methyl Ester

The acetate prepared in step 1 (9.10 g, 0.041 mole) was converted to the acid chloride by dissolving in 50 ml dichloromethane. Dimethylformamide (0.1 ml, catalytic) was added and the reaction mixture cooled in an ice water bath to ₀° C. Oxalyl chloride (25 ml, 0.050 mole) was added drop-wise over 15 minutes. The reaction was then allowed to warm to room temperature and stirred for 1 hour. The reaction was then concentrated in-vacuo and dried under vacuum. The resulting acid chloride was dissolved in 40 ml dichloromethane and added dropwise over 45 minutes to a solution of cyclopropylamine (2.81 g, 0.045 mole) and triethylamine (4.58 g, 0.045 mole) at 0° C. The reaction was then allowed to warm to room temperature and stirred overnight. The sample was diluted with 25 ml dichloromethane and this solution was then washed with 1M HCl (2×), saturated sodium bicarbonate (2×) and saturated sodium chloride. The organic layer was then dried over MgSO₄ and concentrated in-vacuo to give a solid. The product was triturated in 100 ml diethyl ether for 30 minutes, filtered and dried to give a solid. ¹H NMR (300 MHz, CDCl₃) δ 7.64 (d, 2H), 7.54 (d, 2H), 6.49 (bs, 1H), 6.03 (s, 1H), 2.70 (m, 1H), 2.19 (s, 3H), 0.78 (m, 2H), 0.49 (m, 2H).

Step 3—2-(4-Cyanophenyl)-2-hydroxy-N-(cyclopropyl)-acetamide

The amide-acetate prepared in step 2 (6.76 g, 0.0262 mole) was dissolved in 40 ml tetrahydrofuran. To this was added lithium hydroxide monohydrate (1.65 g, 0.0393 mole) dissolved in 15 ml water. The resulting mixture was allowed to stir at room temperature. After 2 hours, 20 ml water and 25 ml ethyl acetate were added. The aqueous layer was separated and extracted with ethyl acetate. The organic layers were combined and washed with saturated sodium chloride. The organic layer was then dried over MgSO4 and concentrated in-vacuo to give the product as a solid. This solid was triturated with diethyl ether filtered and dried to give the product. ¹H NMR (300 MHz, CDCl₃) δ 7.63 (d, 2H), 7.58 (d, 2H), 6.49 (bs, 1H), 5.05 (s, 1H), 2.68 (m, 1H), 0.78 (m, 2H), 0.49 (m, 2H).

Step 4—Preparation of 3-(Cyclohexyl)-Propionic Acid (4-Cyanophenyl)-(Cyclopropylcarbamoyl)-Methyl Ester

The hydroxy amide prepared in step 3 (500 mg, 2.31 mmole) and cyclohexanepropionic acid (397 mg, 2.54 mmole) were mostly dissolved in 20 ml chloroform and 1 ml dimethylformamide. To this reaction was added dimethylaminopyridine (5.0 mg, 0.04 mmole) and then EDCI (756 mg, 2.54 mmole) dissolved in 1 ml chloroform. The reaction was allowed to stir at room temperature for 16 hours. The reaction mixture was then diluted with 20 ml chloroform and transferred to a separatory funnel and washed with 1M HCl (2×), saturated sodium bicarbonate (2×) and saturated sodium chloride. The organic layer was then dried over MgSO4 and concentrated in-vacuo. The compound was then purified by flash chromatography on silica gel (2:1 ethyl acetate/hexane). ¹H NMR (300 MHz, CDCl₃) δ 7.67 (d, 2H), 7.54 (d, 2H), 6.31 (bs, 1H), 6.08 (s, 1H), 2.74 (m, 1H), 2.45 (m, 2H), 1.68 (m, 5H), 1.54 (m, 2H), 1.17 (m, 2H), 0.90 (m, 2H), 0.82 (m, 2H), 0.51 (m, 2H).

### Example 5

### Kallikrein Inhibition Assay

Kallikrein activity was assayed fluorometrically with an aminomethyl coumarin-containing (AMC-containing) substrate according to procedures described in Zimmerman et al., Proc. Natl. Acad. Sci. USA 75:750 (1978), the entire teachings of which are incorporated herein by reference. Specifically, enzyme assays with varying concentrations of the fluorogenic substrate H-Pro-Phe-Arg-AMC were conducted at 24° C in 0.05 M 4-(2-hydroxyethyl)-piperazineethanesulfonic acid (HEPES) buffer, pH 7.5/5% (vol/vol) dimethyl sulfoxide. The final volume was 0.1 ml in each assay. Fluorescence was read after ten minutes at 360 nm (activation wavelength) and 460 nm (emission wavelength).

The results for representative compounds of the present invention are provided below in Table 1.

TABLE 1
Kallikrein Inhibition Data For Compounds of the Present Invention

| Compound Number | Structure | IC50 (μM) |
|---|---|---|
| Compound (11) | 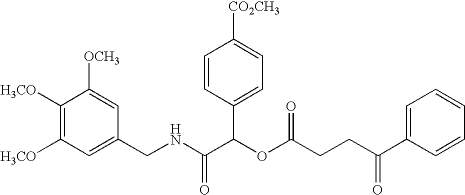 | 0.442 |
| Compound (12) | 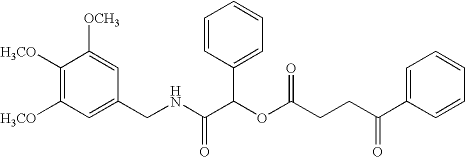 | 0.737 |
| Compound (13) | 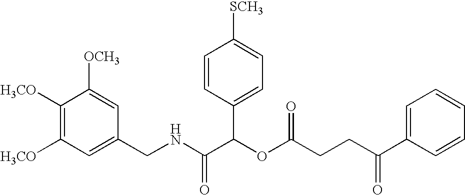 | 0.874 |
| Compound (14) | 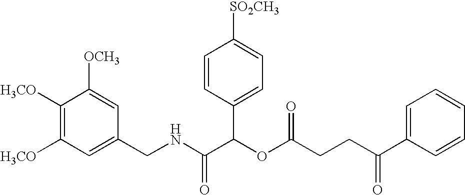 | 1.18 |
| Compound (15) | 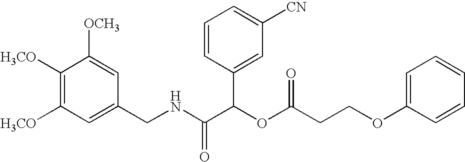 | 0.577 |
| Compound (16) | 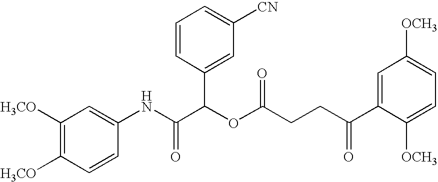 | 0.253 |
| Compound (17) | 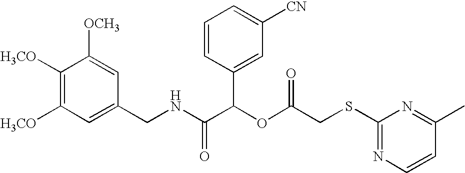 | 0.384 |
| Compound (18) | 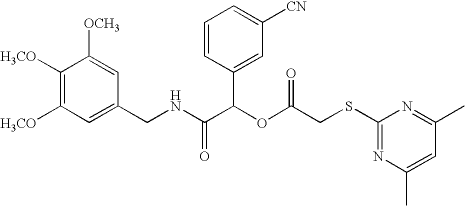 | 6.47 |
| Compound (19) | 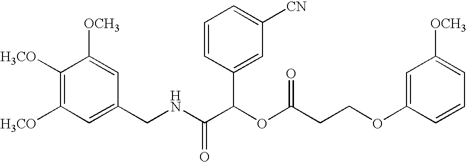 | 1.69 |
| Compound (20) | 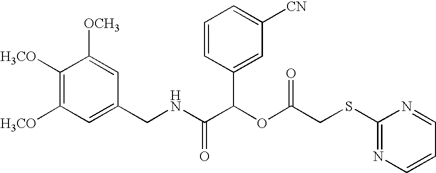 | 1.48 |
| Compound (21) | 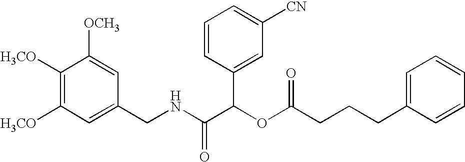 | 1.18 |
| Compound (22) | 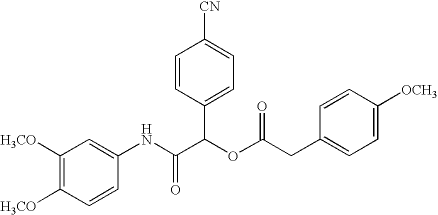 | 0.428 |
| Compound (23) | 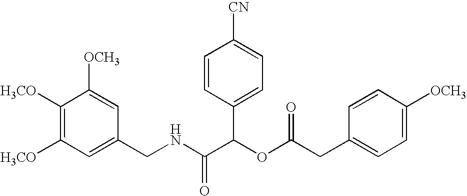 | 0.576 |
| Compound (24) | 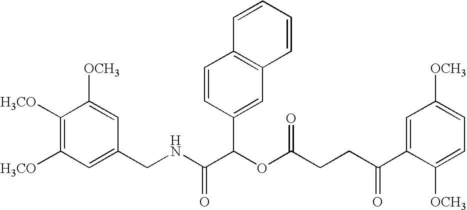 | 0.247 |
| Compound (25) | 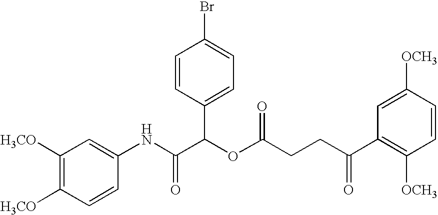 | 0.275 |
| Compound (26) | 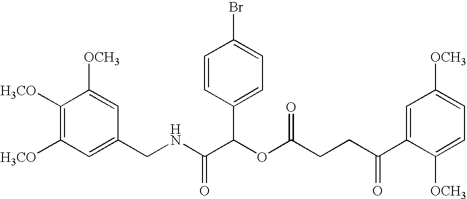 | 0.191 |
| Compound (27) | 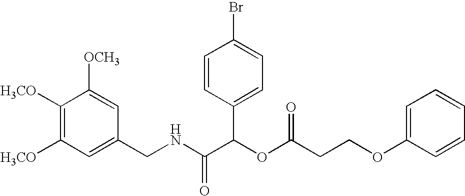 | 0.364 |
| Compound (28) | 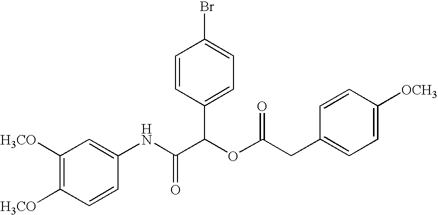 | 0.680 |
| Compound (29) | 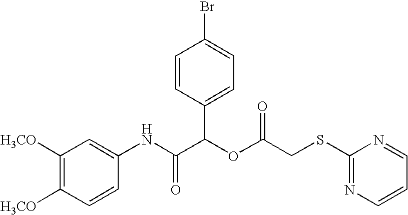 | 0.310 |
| Compound (30) | 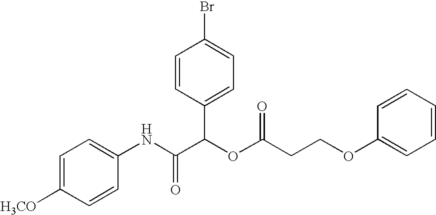 | 0.714 |
| Compound (31) | 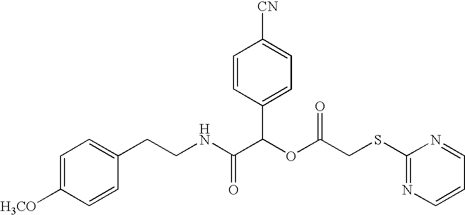 | 0.543 |
| Compound (32) | 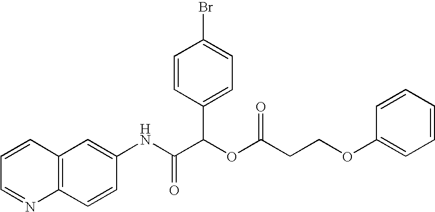 | 1.345 |
| Compound (33) | 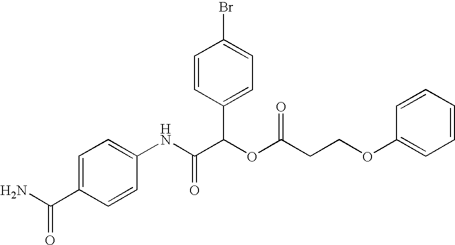 | 0.459 |
| Compound (34) | 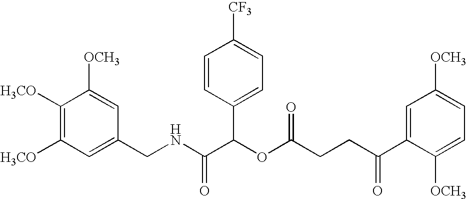 | 0.731 |
| Compound (35) | 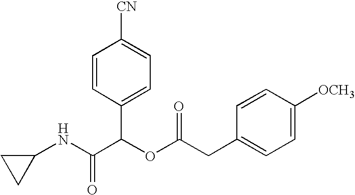 | 0.391 |
| Compound (36) | 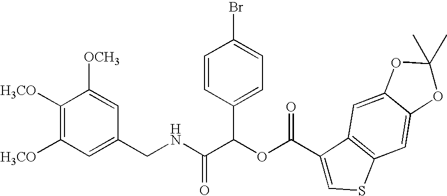 | 3.67 |
| Compound (37) | 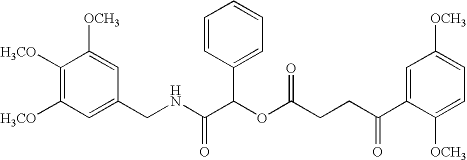 | 1.67 |
| Compound (3) | 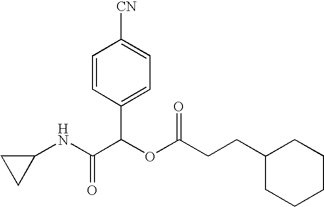 | 1.06 |
| Compound (38) | 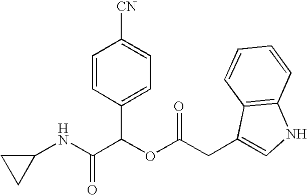 | 3.77 |
| Compound (2) | 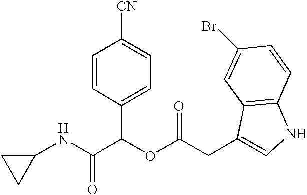 | 0.167 |
| Compound (39) | 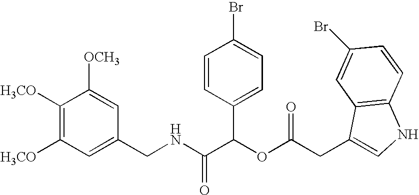 | 2.68 |
| Compound (1) | 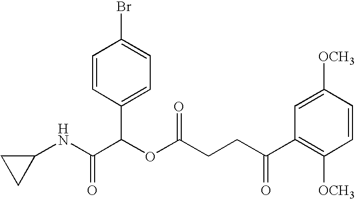 | 0.174 |

### Example 6

### Acetic Acid Writhing Assay

The analgesic activity of a number of compounds of the present invention were tested in an acetic acid writhing assay disclosed in Inoue et al., Arzneimittel Forschung Drug Research 41:228 (1991), the entire teachings of which are incorporated herein by reference. The test substance was administered intraperitoneally (500, 100 and 20 mg/kg) to groups of 3 ICR derived male or female mice weighing 22±2 g one hour before the injection of acetic acid (3%, 10 ml/kg, IP). Reduction in the number of writhes by 50% or more per group of animals observed during the 5-10 minutes period after acetic acid administration, relative to a vehicle-treated control group (challenge with acetic acid), indicates analgesic activity.

The results for the compounds tested are shown below in Table 2.

TABLE 2
Results for Test Compounds in the Acetic Acid Writhing Test

| Treatment | Dose (IP) | | % Reduction of Writhes |
|---|---|---|---|
| Vehicle (1% Cremophor EL) | 20 | ml/kg | 0* |
| (Challenge with acetic acid) | | | |
| Vehicle (1% Cremophor EL) | 20 | ml/kg | ND |
| (No challenge with acetic | | | |
| acid) | | | |
| Compound (24) | 500 | mg/kg | 83 |
| | 100 | mg/kg | 25 |
| | 20 | mg/kg | 0 |
| Compound (26) | 500 | mg/kg | 75 |
| | 100 | mg/kg | 58 |
| | 20 | mg/kg | 25 |
| Compound (27) | 500 | mg/kg | 92 |
| | 100 | mg/kg | 25 |
| | 20 | mg/kg | 8 |
| Compound (29) | 500 | mg/kg | 83 |
| | 100 | mg/kg | 33 |
| | 20 | mg/kg | 17 |

*Vehicle (1% Cremophor EL) caused 12 writhings during the 5-10 minute period after acetic acid administration.
ND: Not determined because no writhing response was seen in the absence of acetic acid.

The four compounds tested showed dose responsive activity and statistically significant responses at the highest dose (500 mg/kg). Compound (26) had significant response at both the 500 and 100 mg/kg doses.

### Example 7

### Carageenan Assay

The assay was performed according to procedures disclosed in Winter et al, Proc. Exper. Biol. Med 111: 544 (1962), the entire teachings of which are incorporated herein by reference. Specifically, the test substance was administered intraperitoneally (300, 100 and 30 mg/kg) to groups of 3 Long Evans derived male or female overnight fasted rats weighing 150±20 grams two hours before right hind paw injection of carageenan (0.1 ml of 1% suspension intraplantar). Hind paw edema, as a measure of inflammation, was measured 3 hours after carageenan administration using a plethysmometer (Ugo Basile Catalog No. 7150) with water cell (25 mm diameter, Catalog No. 7157). Reduction of hind paw edema by 30% or more indicates significant acute anti-inflammatory activity. The results are shown below in Table 3.

TABLE 3
Results For Test Compounds in The Carageenan Assay

| | | | Edema | |
|---|---|---|---|---|
| Treatment | Dose (IP) | | (ml) | Inhibition |
| Vehicle (1% Cremophor in | 5 | ml/kg | 0 | — |
| saline) | | | | |
| (No carageenan administration) | | | | |
| Vehicle (1% Cremophor EL) | 5 | ml/kg | 0.94 | — |
| (With carageenan administration) | | | | |
| Compound (26) | 300 | mg/kg | 0.62 | 34 |
| | 100 | mg/kg | 0.76 | 19 |
| | 30 | mg/kg | 0.97 | 0 |
| Indomethacin (control) | 10 | mg/kg | 0.44 | 53 |

Compound (26) showed dose responsive activity and statistically significant response at the highest dose (300 mg/kg).

### Example 8

### Compound 26 Inhibits Inflammation in a Rat Model of Inflammatory Bowel Disease

Compounds were tested according to procedures described in C M Hogaboam et al., Eur. J. Pharmacol. 309:261 (1996), the entire teachings of which are incorporated herein by reference. Groups of 3 overnight fasted male rats weighing 150±10 grams were used. Distal colitis was induced by intra-colonic instillation of 0.5 ml/rat of DNBS (2,4-dinitrobenzene sulfonic acid, 60 mg/ml in ethanol 30%) after which air (2 ml) was gently injected through the cannula to ensure that the solution remained in the colon. Test compound was administered orally 24 and 2 hours before DNBS instillation and then daily for 5 days in total of 7 doses. The animals were sacrificed 24 hours after the final dose of test compound administration and each colon was removed and weighed. A 30 percent or more (≧30%) inhibition in net increase of colon to 100 grams body weight ratio, relative to vehicle-control-2 group is considered significant.

TABLE 4
Results for test compounds in a rat model of
inflammatory bowel disease.

| | | | | | Inhi- |
|---|---|---|---|---|---|
| | | | | Net | bi- |
| Treatment | Dose (PO)¹ | | Ave.² | Inc.³ | tion⁴ |
| Group 1: Vehicle (1% Cremophor | 10 ml/kg | × 7 | 0.487 | — | — |
| in Dist. Water) (No DNBS | | | | | |
| administration) | | | | | |
| Group 2: Vehicle (1% Cremophor | 10 ml/kg | × 7 | 1.040 | 0.533 | — |
| in Dist. Water) (With DNBS | | | | | |
| administration) | | | | | |
| Group 3: Compound 26 | 75 mg/kg | × 7 | 0.857 | 0.370 | 33 |
| (With DNBS administration) | 20 mg/kg | × 7 | 0.947 | 0.460 | 17 |
| | 4 mg/kg | × 7 | 1.061 | 0.574 | 0 |
| Group 4: Sulfasalazine (With | 500 mg/kg | × 7 | 0.865 | 0.378 | 32 |
| DNBS administration) | | | | | |

¹Parenteral Orally
²Average final weight of the colon in grams
³Net increase in weight (grams) of the colon relative to control (Group 1)
⁴Percent inhibition in net increase of colon weight relative to control (Group 2).

As can be seen from the results in Table 4, Compound 26 showed dose responsive activity and statistically significant response at the highest dose (75 mg/kg).

### Example 9

### Urokinase Assay

Urokinase activity was assayed fluorometrically with an aminomethyl coumarin-containing (AMC-containing) substrate according to procedures described in Zimmerman et al, Proc. Natl. Acad. Sci. USA 75:750 (1978), the entire teachings of which are incorporated herein by reference. Specifically, enzyme assays with varying concentrations of the fluorogenic substrate Cbz-Gly-Gly-Arg-AMC were conducted at 24° C. in 0.05 M 4-(2-hydroxyethyl)-piperazineethanesulfonic acid (HEPES) buffer, pH 7.5/5% (vol/vol) dimethyl sulfoxide; the final volume was 0.1 ml in each assay.

Fluorescence was read after ten minutes at 360 nm (activation wavelength) and 460 nm (emission wavelength).

The results for representative compounds of the present invention are provided below in Table 5.

TABLE 5
Inhibition of urokinase by compound of the present invention
Urokinase Inhibition Data

| Compound Number | Structure | IC₅₀ (μM) |
|---|---|---|
| Compound (40) | 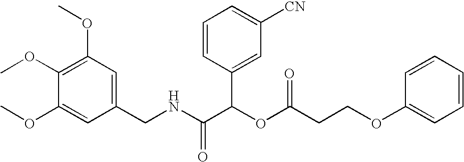 | 6.09 |
| Compound (9) | 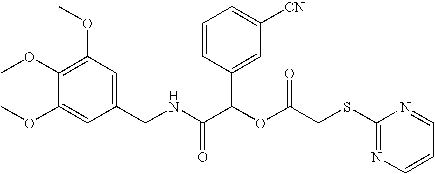 | 0.923 |
| Compound (41) | 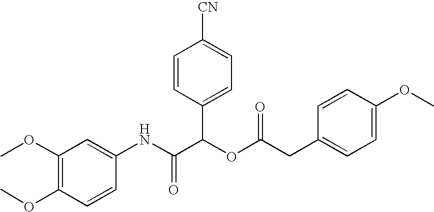 | 2.32 |
| Compound (42) | 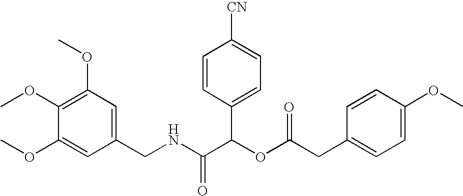 | 1.97 |
| Compound (43) | 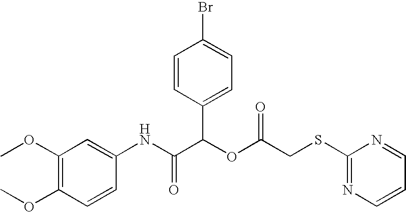 | 3.39 |
| Compound (44) | 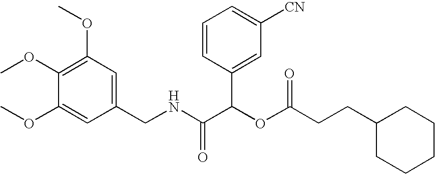 | 1.78 |
| Compound (8) | 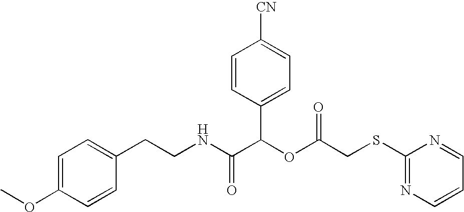 | 0.112 |
| Compound (45) | 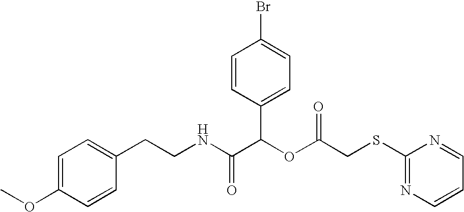 | 1.41 |
| Compound (10) | 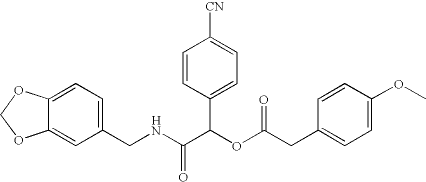 | 0.783 |
| Compound (46) | 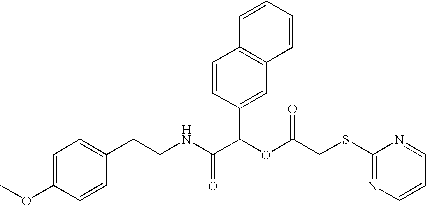 | 2.93 |
| Compound (47) | 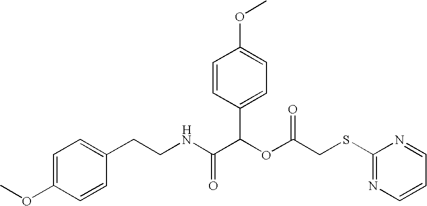 | 2.38 |
| Compound (48) | 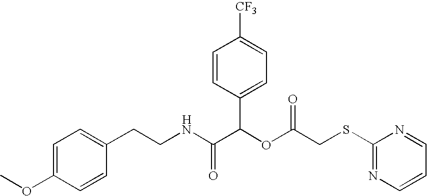 | 5.38 |
| Compound (49) | 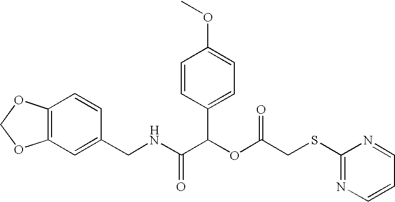 | 1.37 |
| Compound (50) | 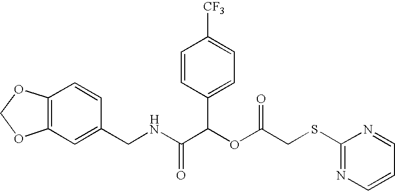 | 3.10 |
| Compound (51) | 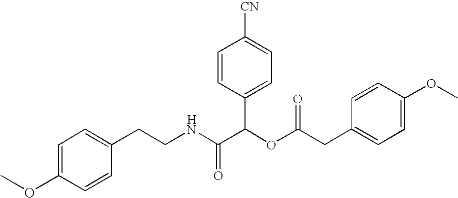 | 1.22 |
| Compound (52) | 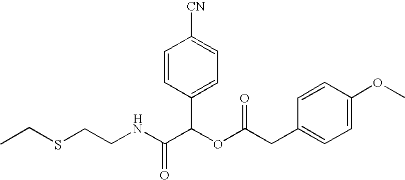 | 0.628 |
| Compound (7) | 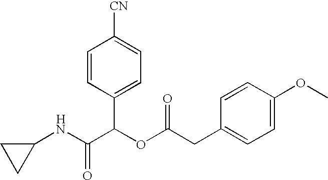 | 0.110 |
| Compound (53) | 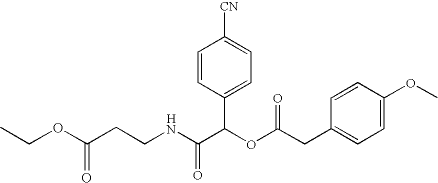 | 1.06 |
| Compound (6) | 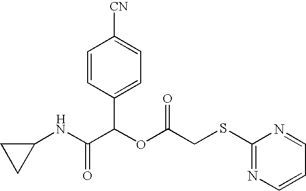 | 0.032 |
| Compound (54) | 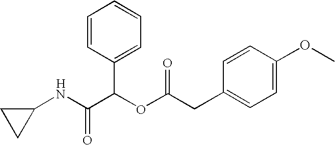 | 0.814 |
| Compound (55) | 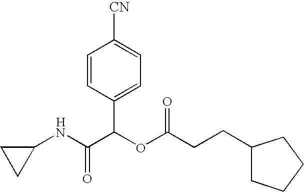 | 0.151 |
| Compound (56) | 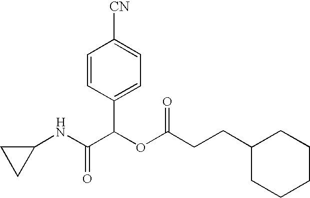 | 0.055 |
| Compound (4) | 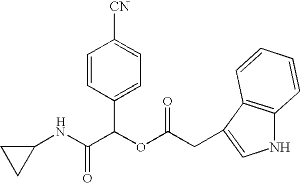 | 0.086 |
| Compound (5) | 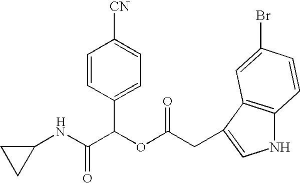 | 0.029 |

As can be seen from this data, compounds of the present invention are effective inhibitors of urokinase.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting kallikrein activity in a subject in need of a such inhibition, said method comprising the step of administering to the subject an effective amount of a compound represented by the following structural formula: 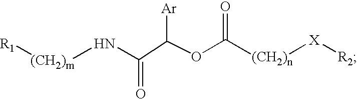
or a pharmaceutically acceptable salt thereof, wherein:
R₁ is a substituted or unsubstituted aryl group or alkyl group;
R₂ is a substituted or unsubstituted aryl group or cycloalkyl group;
Ar is a substituted or unsubstituted aryl group;
X is —CH₂—, —O—, —S— or —CO—;
m is an integer from zero to two; and
n is an integer from 0-2 when X is —O—, —S— or 1-2 when X is —CH₂— or —CO—.

2. The method of claim 1 wherein the subject is being treated with the compound for pain and/or inflammation.

3. The method of claim 1 wherein the subject is being treated with the compound for inflammatory bowel disease.

4. The method of claim 1 wherein the subject is being treated with the compound for rheumatoid arthritis.

5. The method of claim 1 wherein the subject is being treated with the compound for cancer.

6. The method of claim 1 wherein R₁ is cyclopropyl and m is 0.

7. The method of claim 6 wherein Ar is a substituted or unsubstituted phenyl or naphthyl group.

8. The method of claim 7 wherein R₂ is a substituted or unsubstituted phenyl, cyclohexyl or indolyl group.

9. The method of claim 8 wherein Ar is a phenyl group optionally substituted at the three, four and/or five position with methylenedioxy, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl).

10. The method of claim 8 wherein n is 0, X is —CH₂— and R₂ is a substituted or unsubstituted indolyl group.

11. The method of claim 9 wherein n is 2, X is —CO— and R₂ is a substituted or unsubstituted phenyl group.

12. A method of inhibiting kallikrein activity in a subject in need of a such inhibition, said method comprising the step of administering to the subject an effective amount of a compound represented a structural formula selected from: 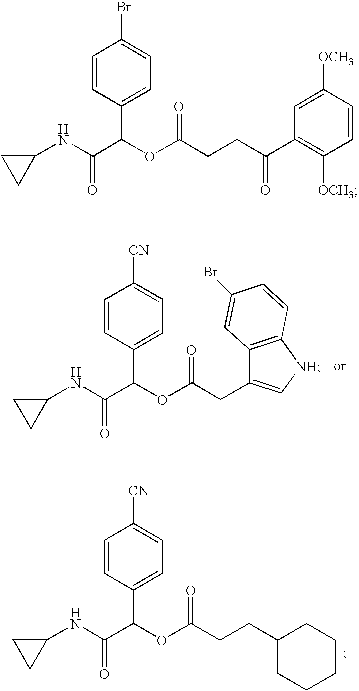
or a pharmaceutically acceptable salt thereof.

13. The method of claim 1 wherein Ar is a substituted or unsubstituted phenyl or naphthyl group.

14. The method of claim 13 wherein R₁ is a substituted or unsubstituted phenyl group.

15. The method of claim 14 wherein R₂ is a substituted or unsubstituted phenyl, pyrimidine, indolyl or benzothienyl group.

16. The method of claim 15 wherein m is 0 or 1; n is 2; and X is —CO—.

17. The method of claim 15 wherein R₁ a is phenyl substituted with one or more groups selected from methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl) and Ar is a phenyl group substituted at the three, four and/or five position with methylenedioxy, —CO—NH₂, —O(C1-C4 alkyl), —F, —Cl, —Br, —CN, C1-C4 alkyl, C1-C4 haloalkyl, —CN, —Br, —Cl, —CF₃, —SO₂(C1-C4 alkyl), —COO(C1-C4 alkyl) or —S(C1-C4 alkyl).

18. The method claim 17 wherein R₂ is a substituted phenyl group.

19. The method of claim 18 wherein R₁ and R₂ are independently phenyl substituted with one or more methoxy groups.

20. The method of claim 19 wherein Ar is group phenyl optionally monosubstituted with —CN or —Br.

21. A method of inhibiting kallikrein activity in a subject in need of a such inhibition, said method comprising the step of administering to the subject an effective amount of a compound represented by the following structural formula: 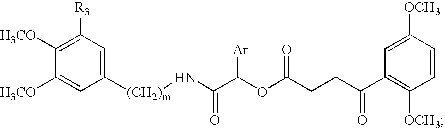
or a pharmaceutically acceptable salt thereof, wherein:
m is 0 or 1; and
R₃ is —H or OCH₃; and Ar is naphthyl or phenyl optionally monosubstituted with —CN or —Br.

22. The method of claim 21 wherein Ar is 2-naphthyl, 4-bromophenyl or 3-cyanophenyl.

23. A method of inhibiting urokinase activity in a subject in need of such inhibition, said method comprising the step of administering to the subject an effective amount of a compound represented by the following structural formula: 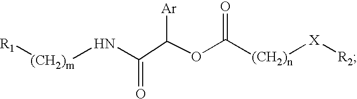
or a pharmaceutically acceptable salt thereof, wherein:
R₁ is a substituted or unsubstituted aryl group or alkyl group;
R₂ is a substituted or unsubstituted aryl group or cycloalkyl group;
Ar is a substituted or unsubstituted aryl group;
X is a —CH₂—, —O—, —S— or —CO—;
m is an integer from zero to two; and
n is an integer from 0-2 when X is —O—, —S— and 1-2 when X is —CH₂— or —CO—.

24. The method of claim 23 wherein the subject is being treated with the compound for cancer.

25. The method of claim 24 wherein R₁ is a cyclopropyl group and m is 0.

26. The method of claim 25 wherein Ar is a substituted or unsubstituted phenyl or naphthyl group.

27. The method of claim 26 wherein R₂ is a substituted or unsubstituted C5-C6 cycloalkyl, phenyl, pyrimidyl or indolyl group.

28. The method of claim 27 wherein Ar is phenyl optionally substituted at the three, four and/or five position with methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl).

29. The method of claim 28 wherein: 1) n is 0, X is —CH₂—, and R₂ is an optionally substituted phenyl or indolyl group; 2) n is 1, X is —S—, and R₂ is an optionally substituted pyrimidyl group; or 3) n is 1, X is —CH₂— and R₂ is a cyclopentyl or cyclohexyl group, wherein the phenyl, pyrimidyl or indolyl group represented by R₂ is optionally substituted with one or more groups selected from methylenedioxy, —CO—NH₂, —O(C1-C4 alkyl), —F, —Cl, —Br, —CN, C1-C4 alkyl, C1-C4 haloalkyl, —CN, —Br, —Cl, —CF₃, —SO₂(C1-C4 alkyl), —COO(C1-C4 alkyl) or —S(C1-C4 alkyl).

30. The method of claim 29 wherein: 1) n is 0, X is —CH₂—, and R₂ is a 4-methoxyphenyl, 3-indolyl or 5-bromo-2-indolyl group; 2) n is 1, X is —S—, and R₂ is 2-pyrimidyl; 3) n is 1, X is —CH₂— and R₂ is a cyclopentyl or cyclohexyl group.

31. A method of inhibiting urokinase activity in a subject in need of a such inhibition, said method comprising the step of administering to the subject an effective amount of a compound represented by the following structural formula: 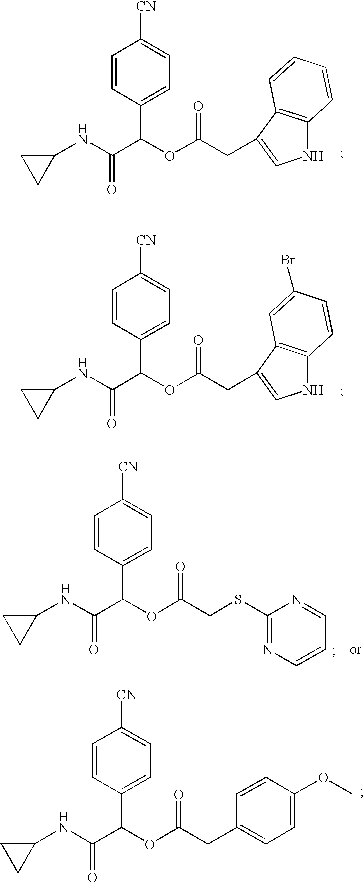
or a pharmaceutically acceptable salt thereof.

32. The method of claim 23 wherein Ar is a substituted or unsubstituted phenyl or naphthyl group.

33. The method of claim 32 wherein R₁ is a substituted or unsubstituted alkyl or phenyl group.

34. The method of claim 32 wherein R₁ is a substituted or unsubstituted phenyl group.

35. The method of claim 34 wherein R₂ is a substituted or unsubstituted pyrimidyl or phenyl group.

36. The method of claim 35 wherein n is 0 or 1; X is —CH₂— or —S—; and m is 1 or 2.

37. The method of claim 33 wherein m is 0; n is 0 or 1; X is —CH₂— or —S—; R₁ is a C1-C4 alkyl group optionally substituted with —O(C1-C4 alkyl), —S(C1-C4 alkyl) or —COO(C1-C4 alkyl); Ar is phenyl optionally substituted with one or more groups selected methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl); and R₂ is phenyl or pyrimidyl optionally substituted with one or more groups selected from methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl).

38. The method of claim 36 wherein Ar is a phenyl group optionally substituted at the three, four and/or five position with methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl); and R₁ is a phenyl group and R₂ is phenyl or pyrimidyl, wherein the phenyl group represented by R₁ and the phenyl and pyrimidyl group represented by R₂ are optionally substituted with one or more groups selected from methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl).

39. The method of claim 38 wherein R₁ is a phenyl group substituted with one or more methoxy groups and/or methylenedioxy groups; Ar is a 4-cyanophenyl group; and R₂ is a pyrimidyl group or phenyl monosubstituted with a methoxy group.

40. The method of claim 39 wherein R₁ is a 4-methoxyphenyl, 3,4-methylenedioxyphenyl, 3,4-dimethoxyphenyl or 3,4,5-trimethoxyphenyl group.

41. A method of inhibiting urokinase activity in a subject in need of a such inhibition, said method comprising the step of administering to the subject an effective amount of a compound represented by a structural formula selected from: 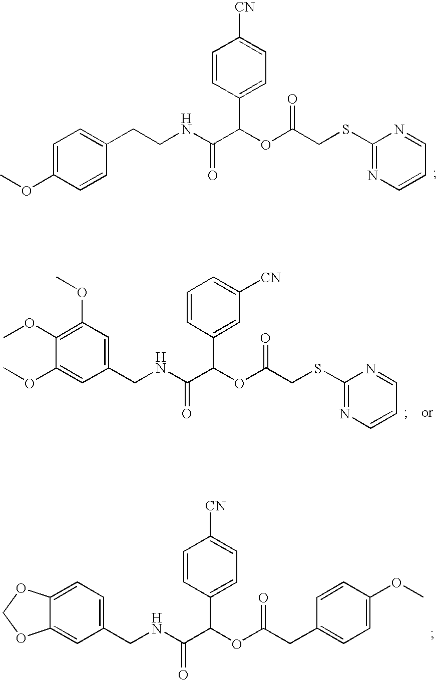
or a pharmaceutically acceptable salt thereof.

42. A compound represented by the following structural formula: 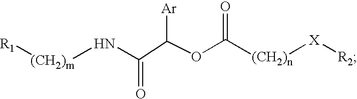
or a pharmaceutically acceptable salt thereof, wherein:
R₁ is a substituted or unsubstituted aryl group or alkyl group;
R₂ is a substituted or unsubstituted aryl group or cycloalkyl group;
Ar is a substituted or unsubstituted aryl group;
X is a —CH₂—, —O—, —S— or —CO—;
m is an integer from zero to two; and
n is an integer from 0-2 when X is —O—, —S— and 1-2 when X is —CH₂— or —CO—,
provided that when Ar is a substituted or unsubstituted phenyl group and X is —CH₂—, then R₁ is a cyclopropyl group or R₂ is a substituted or unsubstituted indolyl, pyrimidinyl, benzothienyl, cyclopentyl or cyclohexyl group.

43. The compound of claim 42 wherein R₁ is cyclopropyl and m is 0.

44. The compound of claim 43 wherein Ar is a substituted or unsubstituted naphthyl group.

45. The compound of claim 44 wherein R₂ is a substituted or unsubstituted C5-C6 cycloalkyl, phenyl, pyrimidyl or indolyl group.

46. The compound of claim 44 wherein Ar is a phenyl group optionally substituted at the three, four and/or five position with methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl), —S(C1-C4 alkyl).

47. The compound of claim 46 wherein: 1) n is 0, X is —CH₂—, and R₂ is an optionally substituted phenyl or indolyl group; 2) n is 1, X is —S—, and R₂ is an optionally substituted pyrimidyl group; 3) n is 1, X is —CH₂— and R₂ is a cyclopentyl or cyclohexyl group; or 4) n is 2, X is —CO— and R₂ is a substituted or unsubstituted phenyl group, wherein the phenyl, pyrimidyl or indolyl group represented by R₂ is optionally substituted with one or more groups selected from methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl).

48. The compound of claim 47 wherein Ar is a phenyl, 4-bromophenyl or 4-cyanophenyl group.

49. A compound represented by a structural formula selected from: 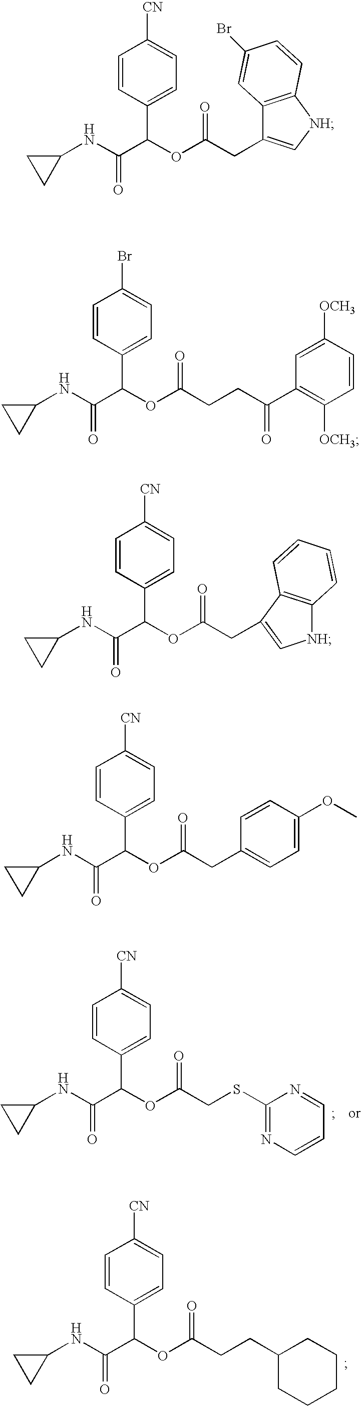
or a pharmaceutically acceptable salt thereof.

50. The compound of claim 42 wherein Ar is a substituted or unsubstituted phenyl or naphthyl group.

51. The compound of claim 50 wherein R₁ is a substituted phenyl or alkyl group.

52. The compound of claim 51 wherein R₁ is a substituted or unsubstituted phenyl group.

53. The compound of claim 52 wherein R₂ is a substituted or unsubstituted phenyl, pyrimidinyl, indolyl or benzothienyl group.

54. The compound of claim 53 wherein Ar is a phenyl group optionally substituted at the three, four and/or five position with methylenedioxy, —CO—NH₂, —O(C1-C4 alkyl), —F, —Cl, —Br, —CN, C1-C4 alkyl, C1-C4 haloalkyl, —SO₂(C1-C4 alkyl), —COO(C1-C4 alkyl) or —S(C1-C4 alkyl); and R₁ is a phenyl group and R₂ is phenyl or pyrimidyl, wherein the phenyl group represented by R₁ and the phenyl and pyrimidyl group represented by R₂ are optionally substituted with one or more groups selected from methylenedioxy, —O(C1-C4 alkyl), —F, —Cl, —Br, —CN, C1-C4 alkyl or C1-C4 haloalkyl.

55. The compound of claim 54 wherein Ar is a phenyl, cyanophenyl or bromophenyl group and R₁ and R₂ are substituted with one or more methoxy groups.

56. The compound of claim 54 wherein Ar is a phenyl, cyanophenyl or bromophenyl group and R₂ is a 2-pyrimidyl group.

57. A compound represented by a structural formula selected from: 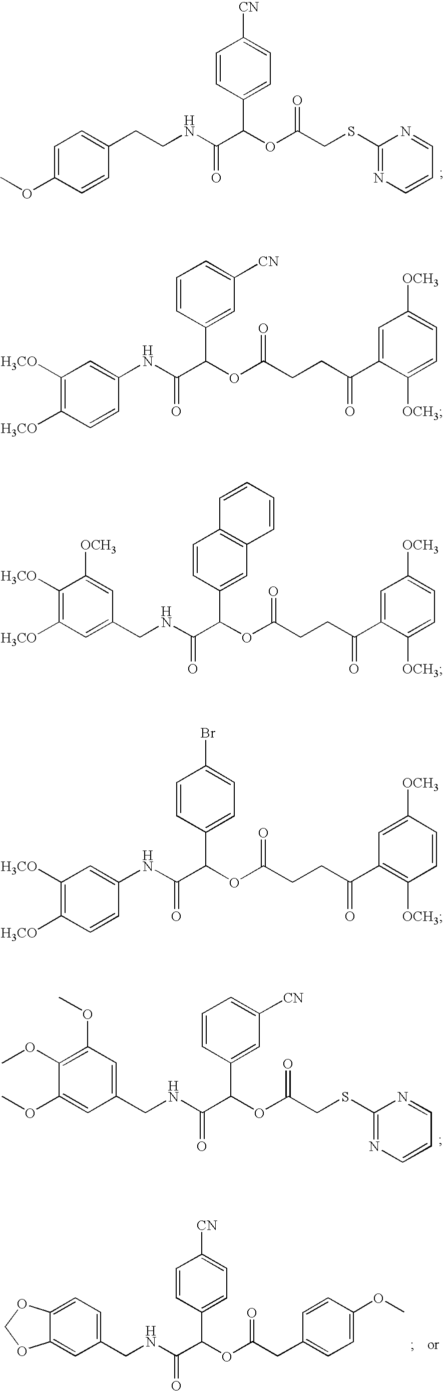 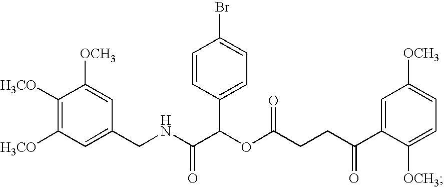
or a pharmaceutically acceptable salt thereof.
